(19) United States
(12) Patent Application Publication
(10) Pub. No.: US 2012/0101282 A1
(43) Pub. Date: Apr. 26, 2012

(54) PROCESS FOR THE PREPARATION OF NICOTINAMIDE DERIVATIVES

(76) Inventors: Pascal Dott, Rixheim (FR); Pius Waldmeier, Wegenstetten (CH)

(21) Appl. No.: 13/271,259

(22) Filed: Oct. 12, 2011

(30) Foreign Application Priority Data
Oct. 22, 2010 (EP) 10188602.6

(51) Int. Cl. C07D 213/64

(52) U.S. Cl. 546/298

(57) ABSTRACT

The present invention relates to a process for the preparation of nicotinamide derivatives of formula I,

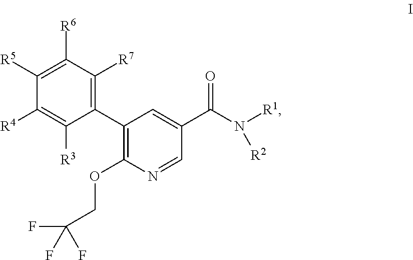

wherein
R¹ to R⁷ are as defined above and to pharmaceutically acceptable salts thereof.

The compounds of formula I are useful for the treatment and/or prophylaxis of diseases which are associated with the modulation of cannabinoid 1 receptors (CB1 receptors) as described in the PCT Publ. WO 2006/106054.

### PRIORITY TO RELATED APPLICATION(S)

This application claims the benefit of European Patent Application No. 10188602.6, filed Oct. 22, 2010, which is hereby incorporated by reference in its entirety.

### FIELD OF THE INVENTION

The present invention relates to a process for the preparation of nicotinamide derivatives of the formula I (described below).

The compounds of formula I are useful for the treatment and/or prophylaxis of diseases which are associated with the modulation of cannabinoid 1 receptors (CB1 receptors) as described in the PCT Publ. WO 2006/106054.

### BACKGROUND OF THE INVENTION

The PCT Publ. WO 2006/106054 discloses various synthetic approaches to the nicotinamide derivatives of formula I in the respective schemes 1 to 7. However, it was found that the overall yield of the above mentioned synthetic approaches were low to moderate based on low yielding reaction steps, formation of several by-products, unselective reactions and incomplete conversions and due to the need of chiral preparative HPLC.

The object of the present invention therefore was to find an alternative synthetic approach which can be applied on a technical scale and which allows the product to be obtained in an excellent yield and purity and without the need of chromatographical purification steps. The object could be achieved with the process of the present invention as outlined below.

### SUMMARY OF THE INVENTION

The present invention relates to a process for the preparation of a nicotinamide derivative of formula I,

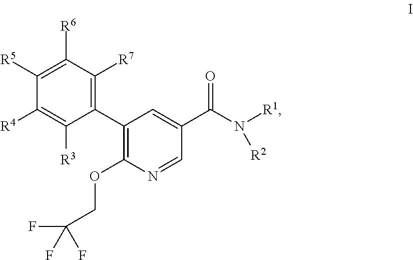

wherein
R¹ is selected from the group consisting of lower hydroxyalkyl, cycloalkyl which is unsubstituted or substituted by hydroxy or lower hydroxyalkyl, and —CH₂—CR⁸R⁹-cycloalkyl;
R⁸ is hydrogen or lower alkyl;
R⁹ is selected from the group consisting of hydrogen, hydroxy and lower alkoxy;
R² is hydrogen;
or R¹ and R² together with the nitrogen atom they are attached to form a piperidinyl ring or a morpholinyl ring;
R³ and R⁷ are hydrogen or halogen; and
R⁴, R⁵ and R⁶ independently from each other are selected from the group consisting of hydrogen, lower alkyl, lower halogenalkyl, lower halogenalkoxy, cyano and halogen and pharmaceutically acceptable salts thereof comprising the step of: a) coupling a 5,6-dihalogenated nicotinic acid derivative of formula III,

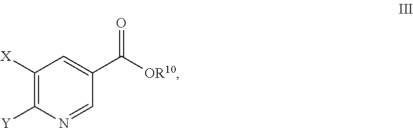

wherein X and Y stand for a halogen atom and R¹⁰ is hydrogen or lower alkyl, with an aryl metal species of formula IV,

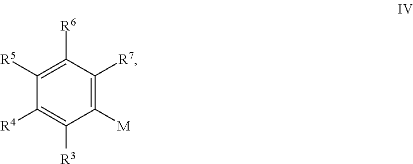

wherein R³ to R⁷ are as defined herein before and M means boronic acid or a boronic acid ester, in the presence of a Pd catalyst under basic conditions to form a 5-aryl substituted nicotinic acid derivative of formula V,

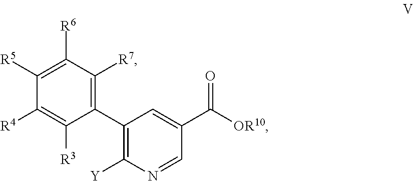

wherein Y, R³, R⁴, R⁵, R⁶, R⁷ and R¹⁰ are as defined herein before.
In an embodiment, the process further comprises the steps of:
b) hydrolyzing a 5-aryl substituted nicotinic acid derivative of formula V wherein R¹⁰ is lower alkyl with a base to form a 5-aryl substituted nicotinic acid derivative of formula VI,

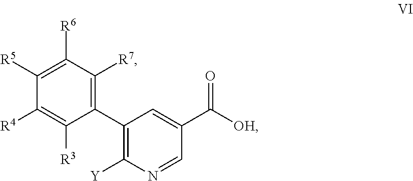

wherein Y, R³, R⁴, R⁵, R⁶ and R⁷ are as defined herein before;
c) introducing a trifluoroethoxy group into the 5-aryl substituted nicotinic acid derivative of formula VI to form a 6-trifluoroethoxy substituted nicotinic acid derivative of formula VII,

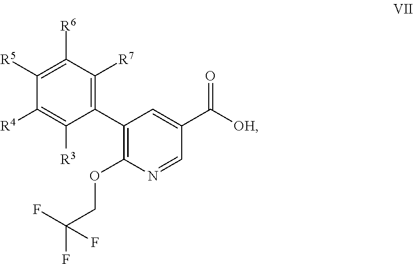

wherein
R³, R⁴, R⁵, R⁶ and R⁷ are as defined herein before;
and
d) forming the nicotinamide derivative of formula I by reacting the 6-trifluoroethoxy substituted nicotinic acid derivative of formula VII with an amine of the formula VIII,

R¹R²NH VIII,

wherein
R¹ and R² are as defined herein before.

### DETAILED DESCRIPTION OF THE INVENTION

The present invention relates to a process for the preparation of nicotinamide derivatives of formula I,

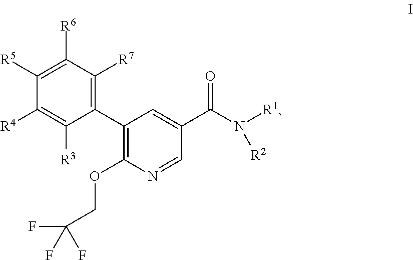

wherein
R¹ is selected from the group consisting of lower hydroxyalkyl, cycloalkyl which is unsubstituted or substituted by hydroxy or lower hydroxyalkyl,
and from —CH₂—CR⁸R⁹-cycloalkyl,
R⁸ is hydrogen or lower alkyl;
R⁹ is selected from the group consisting of hydrogen, hydroxy and lower alkoxy;
R² is hydrogen;
or R¹ and R² together with the nitrogen atom they are attached to form a piperidinyl ring or a morpholinyl ring;
R³ and R⁷ are hydrogen or halogen; and
R⁴, R⁵ and R⁶ independently from each other are selected from the group consisting of hydrogen, lower alkyl, lower halogenalkyl, lower halogenalkoxy, cyano and halogen
and pharmaceutically acceptable salts thereof comprises
a) coupling a 5,6-dihalogenated nicotinic acid derivative of formula III,

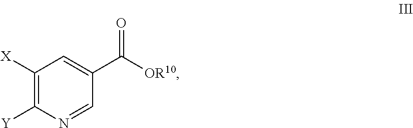

wherein X and Y stand for a halogen atom and R¹⁰ is hydrogen or lower alkyl with an aryl metal species of formula IV,

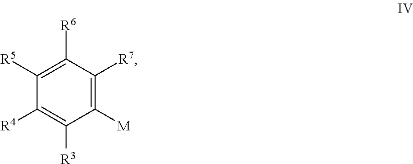

wherein R³ to R⁷ are as defined herein before and M means boronic acid or a boronic acid ester, in the presence of a Pd catalyst under basic conditions to form a 5-aryl substituted nicotinic acid derivative of formula V,

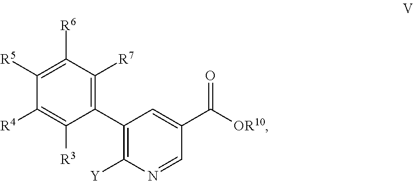

wherein Y, R³, R⁴, R⁵, R⁶, R⁷ and R¹⁰ are as defined herein before;
b) optionally hydrolyzing a 5-aryl substituted nicotinic acid derivative of formula V wherein R¹⁰ is lower alkyl with a base to form a 5-aryl substituted nicotinic acid derivative of formula VI,

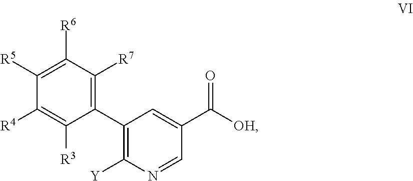

wherein Y, R³, R⁴, R⁵, R⁶ and R⁷ are as defined herein before;
c) introducing a trifluoroethoxy group into the 5-aryl substituted nicotinic acid derivative of formula VI to form a 6-trifluoroethoxy substituted nicotinic acid derivative of formula VII,

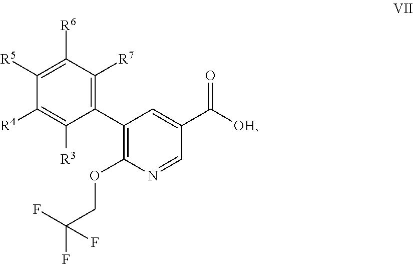

wherein
R³, R⁴, R⁵, R⁶ and R⁷ are as defined herein before;
and
d) forming the nicotinamide derivative of formula I by reacting the 6-trifluoroethoxy substituted nicotinic acid derivative of formula VII with an amine of formula VIII,

R¹R²NH VIII

wherein
R¹ and R² are as defined herein before.

The following definitions are set forth to illustrate and define the meaning and scope of the various terms used to describe the invention herein.

The term “pharmaceutically acceptable salts” embraces salts of the compounds of formula I with inorganic or organic acids such as hydrochloric acid, hydrobromic acid, nitric acid, sulphuric acid, phosphoric acid, citric acid, formic acid, maleic acid, acetic acid, fumaric acid, succinic acid, tartaric acid, methanesulphonic acid, salicylic acid, p-toluenesulphonic acid and the like, which are non toxic to living organisms. Examples of salts with acids are formates, maleates, citrates, hydrochlorides, hydrobromides and methanesulfonic acid salts. In an embodiment, the salt is a hydrochloride.

The term “lower alkyl” refers to a branched or straight-chain monovalent alkyl radical of one to seven carbon atoms, for example one to four carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like, but particularly methyl, ethyl, n-propyl, isopropyl, n-butyl, s-butyl, isobutyl and t-butyl and even more particularly methyl and ethyl.

The term “lower alkoxy” refers to a group R′—O, wherein R′ is lower alkyl as defined above, for example C₁₋₇-alkoxy groups. In an embodiment, the lower alkoxy is a C₁₋₄-alkoxy groups. Examples of lower alkoxy are methoxy, ethoxy, propoxy, isopropoxy, n-butoxy, i-butoxy or t-butoxy.

The term “lower hydroxyalkyl” refers to lower alkyl groups as defined above wherein at least one of the hydrogen atoms of the lower alkyl group is replaced by a hydroxy group, for example C₁₋₇-hydroxyalkyl groups. In an embodiment, the lower hydroxyalkyl is a C₁₋₄-hydroxyalkyl group. Examples of lower hydroxyalkyl groups are 2-hydroxybutyl or 3-hydroxy-2,2-dimethylpropyl.

The term “lower halogenalkyl” refers to lower alkyl groups as defined above wherein at least one of the hydrogen atoms of the lower alkyl group is replaced by halogen as defined below.

In an embodiment, the lower halogenalkyl is a C₁₋₇-halogenalkyl group. In another embodiment, the lower alkyl is a C₁₋₄-halogenalkyl group.

The term “lower halogenalkoxy” refers to lower alkoxy groups as defined above wherein at least one of the hydrogen atoms of the lower alkoxy group is replaced by halogen as defined below. In an embodiment, the lower halogenalkoxy is a C₁₋₇-halogenalkoxy group. In another embodiment, the lower halogenalkoxy is a C₁₋₄-halogenalkoxy group.

The term “cycloalkyl” refers to a monovalent carbocyclic radical of three to seven carbon atoms. In an embodiment, the radical has three to five carbon atoms. This term is exemplified by radicals such as cyclopropyl, cyclobutyl, cyclopentyl, cyclohexyl and cycloheptyl. In an embodiment, the cycloalkyl is a cyclohexyl.

The term “halogen” refers to fluorine, chlorine, bromine and iodine. In a particular embodiment the present invention relates to the preparation of nicotinamide derivatives of the formula I wherein R¹ is cycloalkyl unsubstituted or substituted by hydroxy or lower hydroxyalkyl, R², R³, R⁴ and R⁷ are hydrogen and R⁵ and R⁶ are halogen.

In a further particular embodiment the present invention relates to the preparation of nicotinamide derivatives of the formula I wherein R¹ is 2-hydroxy-cyclohexyl, R², R³, R⁴ and R⁷ are hydrogen and R⁵ and R⁶ are chlorine.

In a still further particular embodiment the present invention relates to the preparation of the optical isomers of 5-(3,4-dichloro-phenyl)-N-2-hydroxy-cyclohexyl)-6-(2,2,2-trifluoro-ethoxy)-nicotinamide of formula Ia,

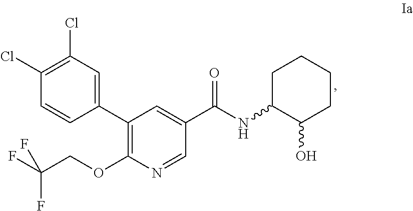

particularly to the isomer 5-(3,4-dichloro-phenyl)-N-((1R,2R)-2-hydroxy-cyclohexyl)-6-(2,2,2-trifluoro-ethoxy)-nicotinamide of formula Ib,

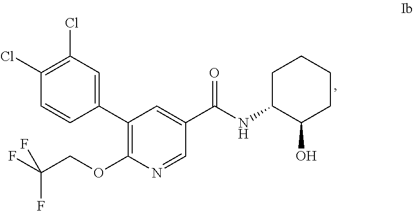

and the isomer 5-(3,4-Dichloro-phenyl)-N-((1S,2R)-2-hydroxy-cyclohexyl)-6-(2,2,2-trifluoro-ethoxy)-nicotinamide of formula Ic,

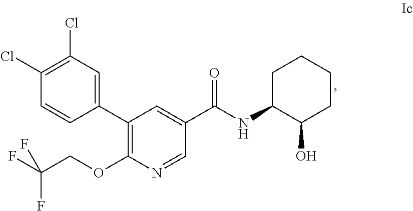

and to pharmaceutically acceptable salts of the isomers.

### Step a

Step a) requires coupling a 5,6-dihalogenated nicotinic acid derivative of the formula III with an aryl metal species of the formula IV in the presence of a Pd catalyst under basic conditions to form a 5-aryl substituted nicotinic acid derivative of the formula V.

The 5,6-dihalogenated nicotinic acid derivative of the formula III are either commercially available or can be manufactured according to the following schemes:

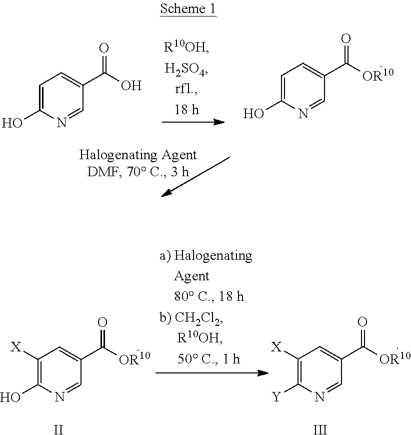

The esterification in the first step is advantageously performed with methanol (R¹⁰ being methyl). This reaction is well known in the literature (see e.g. PCT Publ. WO97/00864 or Oila et al., Tetrahedron Letters 46(6), 967-969 (2005).

The halogenation in the ortho position of the hydroxy group which is characterizing the second step is also known in the literature (see e.g. Meana et al., Synlett (11), 1678-1682 (2003) or Weller et al.; J Org Chem 1983, 48 (25), 4873).

In a particular embodiment the halogenation is an iodination (X being I). Iodosuccinimide may be used as an iodinating agent.

The substitution of the hydroxy group by a halogen in the third step to form the 5,6-dihalogenated nicotinic acid derivative of formula III as a rule is a chlorination which can be effected with phosphorous oxychloride as described in the literature mentioned above.

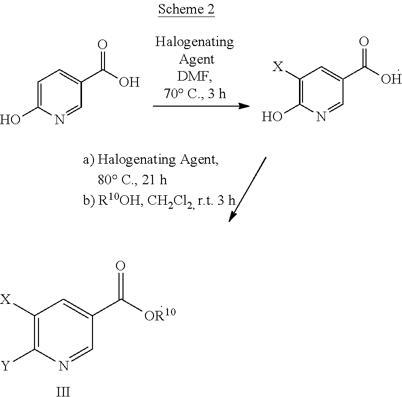

The halogenation in the ortho position of the hydroxy group in the first step is known in the literature (see e.g. Meana et al., Synlett (11), 1678-1682 (2003) or Weller et al.; J Org Chem 1983, 48 (25), 4873). In a particular embodiment the halogenation is a iodination (X being I). Iodosuccinimide may be used as an iodinating agent.

The substitution of the hydroxy group by a halogen in the second step to form the 5,6-dihalogenated nicotinic acid derivative of formula III as a rule is a chlorination which can be effected with phosphorous oxychloride and the optional subsequent esterification can, for example, effected with methanol (R¹⁰ being methyl). This reaction is also described in the literature (see e.g. Signor et al; Gazz Chim Ital 1963, 93, 65 or Wozniak et al.; J Heterocycl Chem 1978, 15, 731).

In a particular embodiment of the present invention the 6-chloro-5-iodo-nicotinic acid methyl ester is selected as advantageous representative for the 5,6-dihalogenated nicotinic acid derivative of formula III.

The subsequent coupling of the 5,6-dihalogenated nicotinic acid derivative of the formula III is performed with an aryl metal species of formula IV

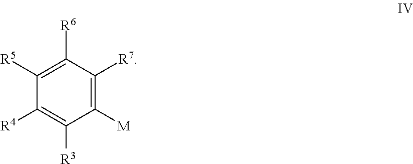

In an embodiment, R³, R⁴ and R⁷ are hydrogen and R⁵ and R⁶ are halogen. In an embodiment, R³, R⁴ and R⁷ are hydrogen and R⁵ and R⁶ are chlorine and M is as above.

3,4-dichlorphenylboronic acid was found to be a favorable aryl metal species of formula IV.

Pd-catalysts which have been found suitable for the coupling can be selected from the group consisting of palladium(II)acetate/triphenylphosphine mixtures, palladium(II)chloride-dppf (1,1′-bis(diphenylphosphino)ferrocene) and palladium(II)chloride bis(triphenylphosphino).

The basic conditions necessary for the coupling can be achieved with the presence of a base selected from a tertiary amine and an alkali carbonate, for example sodium carbonate.

As a rule the reaction can be performed in the presence of an organic solvent, such as an aromatic hydrocarbon like toluene, or toluene/water, DMF, methanol, or methanol/water, at a reaction temperature of 20° C. to 110° C., for example from 70° C. to 90° C.

The resulting 5-aryl substituted nicotinic acid derivative of the formula V can be isolated following methods known to the skilled in the art.

### Step b)

Step b) optionally requires hydrolyzing a 5-aryl substituted nicotinic acid derivative of the formula V wherein R¹⁰ is lower alkyl with a base to form a 5-aryl substituted nicotinic acid derivative of formula VI.

Advantageously the 5-aryl substituted nicotinic acid derivative of formula V obtained in step a) is not isolated and is in situ subjected to the hydrolysis in step b) for the formation of the 5-aryl substituted nicotinic acid derivative of formula VI.

In an embodiment the 5-aryl substituted nicotinic acid derivative of formula V is the 6-Chloro-5-(3,4-dichloro-phenyl)-nicotinic acid methylester which is hydrolyzed to form the 6-Chloro-5-(3,4-dichloro-phenyl)-nicotinic acid as 5-aryl substituted nicotinic acid derivative of the formula VI.

The hydrolysis can usually be performed with a base selected from an alkali hydroxide, in a mixture of a suitable organic solvent such as tetrahydrofuran and water.

Suitable alkali hydroxides are selected from the group consisting of lithium-, sodium- and potassium hydroxide. In an embodiment, an aqueous solution of lithium hydroxide is used.

The reaction is as a rule performed in the same organic solvent as used for the previous coupling step at a reaction temperature of 0° C. to 60° C. In an embodiment, the reaction temperature is from 10° C. to 30° C.

The 5-aryl substituted nicotinic acid derivative of formula VI can be isolated following methods known to the skilled in the art e.g. by acidifying the reaction mixture, by exchanging the solvent towards a lower boiling solvent like ethanol and by filtering off the crystals obtained.

### Step c)

Step c) requires introducing a trifluoroethoxy group into the 5-aryl substituted nicotinic acid derivative of the formula VI to form a 6-trifluoroethoxy substituted nicotinic acid derivative of formula VII.

In an embodiment the 6-trifluoroethoxy substituted nicotinic acid derivative of formula VII obtained in step c) is the 5-(3,4-Dichloro-phenyl)-6-(2,2,2-trifluoro-ethoxy)-nicotinic acid.

The introduction of the trifluoroethoxy group in this step can be effected with 2,2,2-trifluoroethanol in the presence of a base and an organic solvent at a reaction temperature between 20° C. to 150° C., particularly between 60° C. and 100° C.

Suitable bases are alkali hydroxides, such as lithium-, sodium- and potassium hydroxide. In an embodiment, the base is lithium hydroxide or an organic base selected from diazabicycloundecen or from triazabicyclodecene.

Suitable organic solvents are for instance tetrahydrofuran, DMF and NMP.

The isolation of the 6-trifluoroethoxy substituted nicotinic acid derivative of formula VII from the reaction mixture can follow methods known to the skilled in the art via extractive work up and crystallization due to solvent exchange.

### Step d)

Step d) requires forming the nicotinamide derivative of formula I by reacting the 6-trifluoroethoxy substituted nicotinic acid derivative of formula VII with an amine of the formula VIII.

In an embodiment, an amine of formula VIII is selected, wherein R¹ is cycloalkyl unsubstituted or substituted by hydroxy or lower hydroxyalkyl and R² is hydrogen. In an embodiment, R¹ is 2-hydroxy cyclohexyl and R² is hydrogen.

Reactions for forming an amide bond are well known in the art.

As a rule a coupling agent is employed to affect the transition.

Suitable coupling agents are oxalyl chloride, N,N′-carbonyl-diimidazole (CDI), N,N′-dicyclohexylcarbodiimide (DCC), 1-(3-dimethylaminopropyl)-3-ethylcarbodiimide hydrochloride (EDCI), 1-[bis(dimethylamino)-methylene]-1H-1,2,3-triazolo[4,5-b]pyridinium-3-oxide hexafluorophosphate (HATU), 1-hydroxy-1,2,3-benzotriazole (HOBT) and O-benzotriazol-1-yl-N,N,N′,N′-tetramethyluronium tetrafluoroborate (TBTU).

Advantageously oxalyl chloride is used as coupling agent to form the respective acid chloride of the 6-trifluoroethoxy substituted nicotinic acid derivative of formula VII.

The acid chloride formation can take place in the presence of a suitable organic solvent like tetrahydrofuran or methyl tetrahydrofuran at a reaction temperature of 0° C. to 120° C.

Thereafter the coupling with the amine of formula VIII can take place, usually in the presence of a base and an organic solvent at a reaction temperature of 10° C. to 30° C.

In an embodiment, the amide formation may be effected in the presence of a coupling agent and an organic solvent at a reaction temperature of 0° C. to 120° C.

Suitable bases are alkali hydroxides, such as lithium-, sodium- or potassium hydroxide. For example, an aqueous solution of sodium hydroxide may be used.

As a rule the same solvent as used for the acid chloride formation is used for the coupling with the amine.

Isolation of the desired nicotinamide derivative of formula I can happen following methods known to the skilled in the art, e.g. by crystallization of the product in a suitable solvent such as in ethanol.

### EXAMPLES

### Abbreviations

DMF N,N-dimethylformamide
DMSO Dimethylsulfoxide
NMP N-methylpyrrolidone
THF Tetrahydrofuran

### Example 1

### 6-Hydroxy-nicotinic acid methyl ester

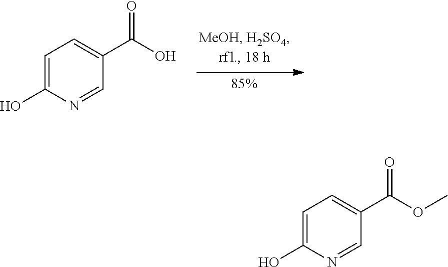

A suspension of 5-hydroxynicotinic acid (200 g, 1438 mmol) in methanol (1.0 l) was treated drop wise over 25 min with sulfuric acid (84 ml, 1505 mmol, exothermic!), heated to reflux and stirred at this temperature for 18 h. The yellow solution was cooled to ca. 30° C. and the solvent evaporated under reduced pressure (ca. 50-100 mbar) until a residual volume of ca. 600 ml. The solvent of the formed suspension was exchanged by water keeping the volume of ca. 600 ml. The suspension was stirred for 1 h at room temperature. The crystals were filtered, washed with water (50 ml) and dried to isolate the product in 68% yield. MS (GC_Split): 153 (M, 64%), 122 (100%), 94 (36), 66 (14).

### Example 2

### 6-Hydroxy-5-iodo-nicotinic acid methyl ester

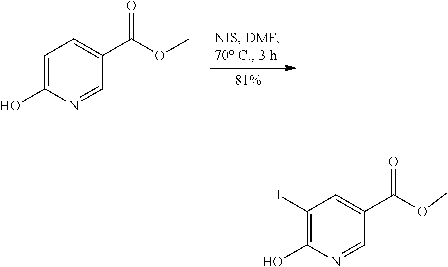

A solution of 6-hydroxy-nicotinic acid methyl ester (100.0 g, 653.0 mmol) and N-Iodosuccinimide (162.0 g, 720.1 mmol) in DMF (500 ml) was heated to 70° C. and stirred for 3 h. The reaction mixture was cooled within 20 min to room temperature and treated within 30 min with a solution of sodium thiosulfate (30.0 g, 189.7 mmol) in water (600 ml). The formed suspension was stirred for 30 min at room temperature, the crystals filtered, washed with water (500 ml) and dried to isolate the desired product in 81% yield. MS (pos): 302 (M+H⁻, 5%), 280 (M+H⁻, 100%).

### Example 3

### 6-Chloro-5-iodo-nicotinic acid methyl ester

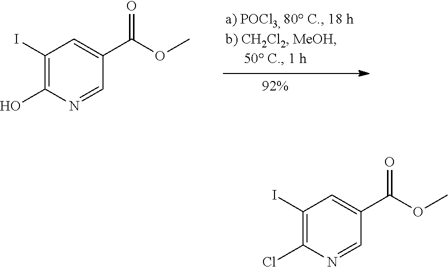

To a suspension of 6-hydroxy-5-iodo-nicotinic acid methyl ester (150.0 g, 538.0 mmol) in acetonitrile was added at room temperature phosphorus oxychloride (123.0 g, 805.0 mmol). The suspension was heated to reflux and stirred for 18 h. After cooling to 50° C., methanol (70.1 g, 2.19 mol) was added and the reaction mixture was stirred at this temperature for 1 h. The formed suspension was cooled to room temperature and treated with water (1.1 l). The crystals were filtered, washed with water (600 ml) and dried to isolate the product in 92% yield. MS (GC-Split): 297 (M, 84%), 266 (100), 238 (36), 111 (56).

### Example 4

### 6-Hydroxy-5-iodo-nicotinic acid

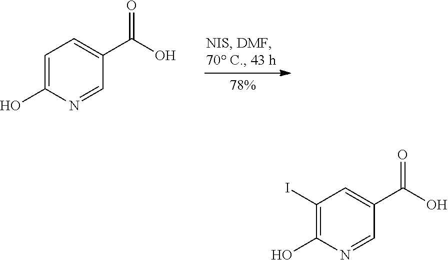

A solution of 6-hydroxy-nicotinic acid (1.0 g, 7.2 mmol) and N-Iodosuccinimide (1.8 g, 7.9 mmol) in DMF (8.0 ml) was heated to 70° C. and stirred for 4 h. The reaction mixture was cooled to room temperature and the suspension treated with a solution of sodium thiosulfate 0.1 N (50.0 ml, 5.0 mmol) and water (15 ml). The formed suspension was stirred for 3 days at room temperature, the crystals filtered, washed with water and dried to isolate the desired product in 78% yield. MS (mixed scan): 264 (M−H⁻, 100%).

### Example 5

### 6-Chloro-5-iodo-nicotinic acid methyl ester

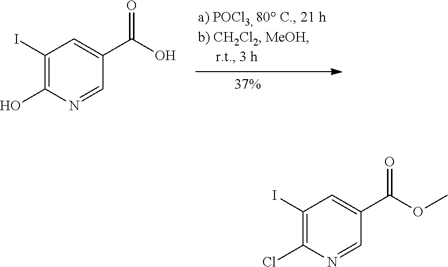

To a suspension of 6-hydroxy-5-iodo-nicotinic acid (200.0 mg, 755 μmmol) in toluene (1.0 ml) was added at room temperature phosphorus oxychloride (289.0 mg, 1.9 mmol). The suspension was heated to 80° C. and stirred for 21 h. After cooling to room temperature, methanol (101 μl, 2.49 mmol) was added and the reaction mixture was stirred at this temperature for 3 h. The reaction mixture was treated with toluene (10 ml) and extracted 3 times with water (total 30 ml) to isolate after evaporation of the organic solvent the crude product in 37% yield. MS (GC-Split): 297 (M, 84%), 266 (100), 238 (36), 111 (56).

### Example 6

### 6-Chloro-5-(3,4-dichloro-phenyl)-nicotinic acid

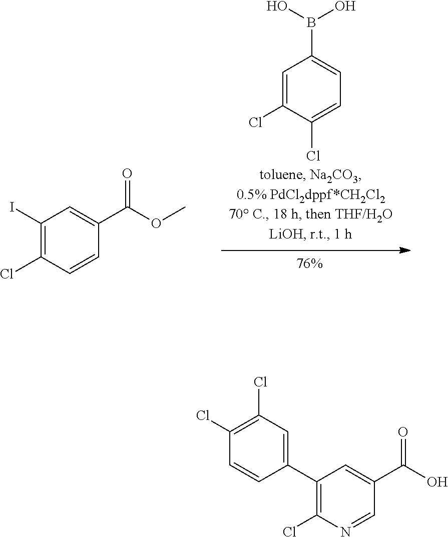

A suspension of 6-chloro-5-iodo-nicotinic acid methyl ester (20.0 g, 67.2 mmol), 3,4-dichlorophenylboronic acid (13.3 g, 69.9 mmol) and 1,1′-bis(diphenylphosphino)ferrocene palladium(II) dichloride dichloromethane complex (275.0 mg, 336 mol) in toluene (120 ml) was treated at room temperature with a solution of sodium carbonate (14.3 g, 134.0 mmol) in water (60.0 ml). The suspension was heated to 70° C. and stirred for 18 h. After cooling to 60° C. the phases were separated and the organic phase was washed at 60° C. with water (60 ml). The combined organic phase was treated with a solution of lithium hydroxide monohydrate (5.7 g, 136 mmol) in water (65 ml) and stirred for 2 h at 60° C. The solution was cooled to room temperature, treated with water (60 ml) and within 10 min with HCl (18.0 ml, 138 mmol). The solvent of the formed suspension was exchanged under constant volume with ethanol (ca. 400 ml) at 45° C. and 100 to 250 mbar and stirred for 17 h at room temperature. The crystals were filtered, washed with water (100 ml) and dried to isolate the product in 76% yield. MS (pos): 306 (M+H⁻, 31%), 304 (M+H⁻, 85%), 302 (M+H', 100%).

### Example 7

### 5-(3,4-Dichloro-phenyl)-6-(2,2,2-trifluoro-ethoxy)-nicotinic acid

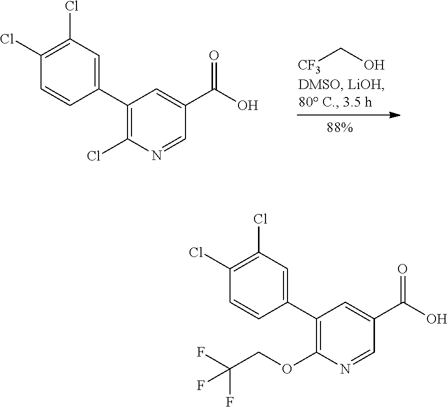

A suspension of 6-chloro-5-(3,4-dichloro-phenyl)-nicotinic acid (70.0 g, 231.4 mmol) in DMSO (350.0 ml) was treated at room temperature in one part with LiOH (14.1 g, 578.5 mmol, exothermic->31° C.). To the suspension was added within 10 min 2,2,2-trifluoroethanol (46.2 g, 462.8 mmol, exothermic->35° C.). The reaction mixture was heated to 80° C. and stirred for 4 h. After cooling to room temperature, water (700 ml) was added within 15 min, followed by the addition of dicalite (35.0 g). The suspension was stirred over night, filtered and the residue washed with water (140 ml). To the filtrate THF (560 ml) was added and within 15 min HCl % (75.0 ml, 575.3 mmol) to adjust the pH<2. After stirring for 5 min at room temperature the phases were separated, the organic phase was treated with THF (160 ml) and filtered over QuadraPure™ MPA (Sigma Aldrich) (0.7 g) and washed with THF (50 l). The solvent of the mixture was exchanged with ethanol (700 ml) keeping the volume constant. The formed suspension was stirred for 1 h at 0° C., filtered and the crystals washed with ethanol (70 ml). After drying the product was isolated in 88% yield. MS (TurboSpray): 366 (M−H, 65%), 364 (M−H, 100%).

### Example 8

### 5-(3,4-Dichloro-phenyl)-N-((1R,2R)-2-hydroxy-cyclohexyl)-6-(2,2,2-trifluoro-ethoxy)-nicotinamide

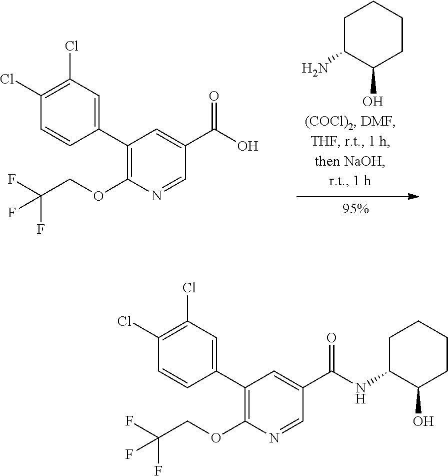

To a solution of 5-(3,4-dichloro-phenyl)-6-(2,2,2-trifluoro-ethoxy)-nicotinic acid (600 g, 1.64 mmol) in a mixture of THF (3.6 l) and DMF (4.0 ml) was added at room temperature within 1 h oxalyl chloride (215.0 ml, 2.46 mol). The reaction mixture was stirred for 1 h at room temperature (acid chloride formation).

A solution of (1R,2R)-2-aminocyclohexanol hydrochloride (298.0 g, 1.97 mol) in a mixture of THF (2.4 l) and water (2.4 l) was treated at room temperature with NaOH (759.0 ml, 8.19 mmol) and stirred for 1 h. The biphasic mixture was heated to ca. 38° C., treated at this temperature within 45 min with the above described acid chloride and stirred for 45 min at ca. 38° C. After addition of ethanol (2.19 l) and stirring for 5 min, water (4.8 l) was added. The mixture was heated to 60° C. and the organic solvent exchanged with ethanol (9.6 l) under reduced pressure keeping the total volume constant. The formed suspension was stirred for 3 h at room temperature, filtered and the crystals washed with a mixture of ethanol (2.1 l) and water 2.1 l) followed by water (3.0 l). After drying the product was isolated in 95% yield. MS (TurboSpray): 465 (M+H⁺, 70%), 463 (M+H⁺, 100%).

1. A process for the preparation of a nicotinamide derivative of formula I, 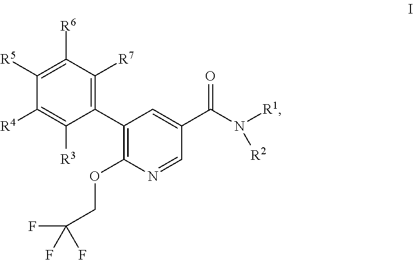
wherein
R¹ is selected from the group consisting of lower hydroxyalkyl, cycloalkyl which is unsubstituted or substituted by hydroxy or lower hydroxyalkyl, and —CH₂—CR⁸R⁹-cycloalkyl;
R⁸ is hydrogen or lower alkyl;
R⁹ is selected from the group consisting of hydrogen, hydroxy and lower alkoxy;
R² is hydrogen;
or R¹ and R² together with the nitrogen atom they are attached to form a piperidinyl ring or a morpholinyl ring;
R³ and R⁷ are hydrogen or halogen; and
R⁴, R⁵ and R⁶ independently from each other are selected from the group consisting of hydrogen, lower alkyl, lower halogenalkyl, lower halogenalkoxy, cyano and halogen
and pharmaceutically acceptable salts thereof comprising the step of: a) coupling a 5,6-dihalogenated nicotinic acid derivative of formula III, 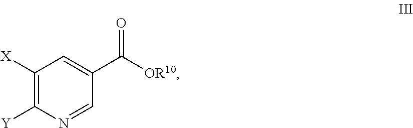
wherein X and Y stand for a halogen atom and R¹⁰ is hydrogen or lower alkyl,
with an aryl metal species of formula IV, 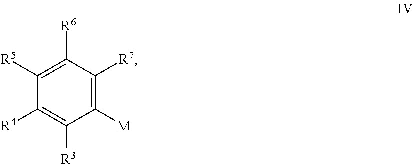
wherein R³ to R⁷ are as defined herein before and M means boronic acid or a boronic acid ester, in the presence of a Pd catalyst under basic conditions to form a 5-aryl substituted nicotinic acid derivative of formula V, 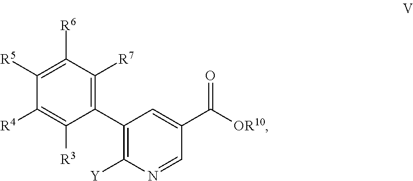
wherein Y, R³, R⁴, R⁵, R⁶, R⁷ and R¹⁰ are as defined herein before.

2. A process according to claim 1, wherein R³, R⁴ and R⁷ are hydrogen and R⁵ and R⁶ are halogen.

3. A process according to claim 2, wherein R³, R⁴ and R⁷ are hydrogen and R⁵ and R⁶ are chlorine.

4. A process according to claim 1, wherein the Pd catalyst is selected from the group consisting of complexes of palladium(II)acetate/triphenylphosphine mixtures, palladium(II)chloride-dppf (1,1′-bis(diphenylphosphino)ferrocene) and palladium(II)chloride bis(triphenylphosphino).

5. A process according to claim 1, wherein the basic conditions for the coupling are achieved with the presence of a base selected from a tertiary amine and an alkali carbonate.

6. A process according to a claim 1, wherein the coupling is performed in the presence of an organic solvent at a reaction temperature of 20° C. to 110° C.

7. A process according to claim 1 further comprising the steps of:
b) hydrolyzing a 5-aryl substituted nicotinic acid derivative of formula V wherein R¹⁰ is lower alkyl with a base to form a 5-aryl substituted nicotinic acid derivative of formula VI, 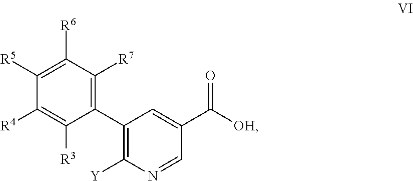
wherein Y, R³, R⁴, R⁵, R⁶ and R⁷ are as defined herein before;
c) introducing a trifluoroethoxy group into the 5-aryl substituted nicotinic acid derivative of formula VI to form a 6-trifluoroethoxy substituted nicotinic acid derivative of formula VII, 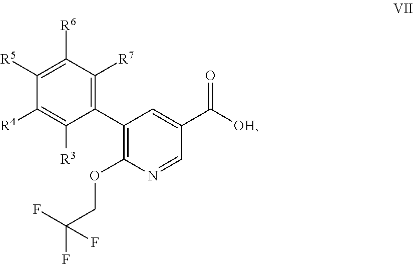
wherein
R³, R⁴, R⁵, R⁶ and R⁷ are as defined herein before;
and
d) forming the nicotinamide derivative of formula I by reacting the 6-trifluoroethoxy substituted nicotinic acid derivative of formula VII with an amine of formula VIII,
R¹R²NH VIII,
wherein
R¹ and R² are as defined herein before.

8. A process according to claim 7, wherein the hydrolysis in step b) is performed with an alkali hydroxide.

9. A process according to claim 7, wherein the 5-aryl substituted nicotinic acid derivative of formula V obtained from step a) is not isolated and is in situ subjected to the hydrolysis in step b) for the formation of the 5-aryl substituted nicotinic acid derivative of formula VI.

10. A process according to claim 7, wherein the introduction of the trifluoroethoxy group in step c) is effected with 2,2,2-trifluoroethanol in the presence of a base and an organic solvent at a reaction temperature between 20° C. to 150° C.

11. A process according to claim 10, wherein the base is selected from an inorganic base selected from an alkali hydroxide or from an organic base that is diazabicycloundecen or triazabicyclodecene.

12. A process according to claim 7, wherein R¹ is cycloalkyl unsubstituted or substituted by hydroxy or lower hydroxyalkyl and R² is hydrogen.

13. A process according to claim 12, wherein R¹ is 2-hydroxy cyclohexyl and R² is hydrogen.

14. A process according to claim 12, wherein the amide formation is effected in the presence of a coupling agent and an organic solvent at a reaction temperature of 0° C. to 120° C.

15. A process according to claim 14, wherein the coupling agent is selected from the group consisting of oxalyl chloride, N,N′-carbonyl-diimidazole (CDI), N,N′-dicyclohexylcarbodiimide (DCC), 1-(3-dimethylaminopropyl)-3-ethylcarbodiimide hydrochloride (EDCI), 1-[bis(dimethylamino)-methylene]-1H-1,2,3-triazolo[4,5-b]pyridinium-3-oxide hexafluorophosphate (HATU), 1-hydroxy-1,2,3-benzotriazole (HOBT) and O-benzotriazol-1-yl-N,N,N′,N′-tetramethyluronium tetrafluoroborate (TBTU).
